(19) United States
(12) Patent Application Publication
(10) Pub. No.: US 2012/0251384 A1
(43) Pub. Date: Oct. 4, 2012

(54) MODULAR DRY HEAT STERILIZER

(76) Inventors: Robert S. Sedlacek, Lake Wales, FL (US); Steven M. Niemi, Lincoln, MA (US)

(21) Appl. No.: 13/508,456

(22) PCT Filed: Nov. 4, 2010
(86) PCT No.: PCT/US10/55412

§ 371 Date: Jun. 19, 2012

Related U.S. Application Data
(60) Provisional application No. 61/258,842, filed on Nov. 6, 2009.

(51) Int. Cl. A61L 2/04

(52) U.S. Cl. 422/22; 422/307

(57) ABSTRACT

A method and an apparatus for sterilizing objects, such as animal cages, are disclosed. The apparatus includes a housing that defines a sterilizing chamber for the objects. The apparatus includes a first air inlet in fluid communication with the sterilizing chamber, and a first air outlet in fluid communication with the sterilizing chamber. The first air inlet and the first air outlet are structured to be placed in fluid communication with a source of dry heat. A second air inlet is in fluid communication with the sterilizing chamber and a source of filtered air. A second air outlet in fluid communication with the sterilizing chamber and an exterior space outside of the sterilizing chamber. Dry heat flows in the first air inlet, over the objects in the sterilizing chamber, and out the first air outlet. Dampers are then closed to seal off the first air inlet and the first air outlet. After sterilization, dampers are opened in the second air inlet and the second air outlet, and cooling filtered air is directed in the second air inlet, over the objects in the sterilizing chamber, and out the second air outlet. Dampers can then be closed to seal off the second air inlet and the second air outlet to avoid contamination of the objects.

### CROSS-REFERENCES TO RELATED APPLICATIONS

This application claims priority based on U.S. Provisional Patent Application No. 61/258,842 filed Nov. 6, 2009.

### STATEMENT REGARDING FEDERALLY SPONSORED RESEARCH

Not Applicable.

### BACKGROUND OF THE INVENTION

1. Field of the Invention

This invention relates to a method and an apparatus for sterilizing objects. In particular, the invention relates to a modular dry heat sterilizer for animal cages.

2. Description of the Related Art

Academic, government and for-profit institutions usually use mice in the course of their research, teaching, and testing activities. Indeed, mice are the most popular animal used in biomedical research and regulatory testing, accounting for more than 95% of all laboratory mammals in developed nations. Due to the continued growth of pharmaceutical and medical research and established means to genetically modify laboratory mice, their use will continue to grow. This growth will be in the form of more mice, higher value mice, and more sophisticated analyses of those mice.

Mouse cages are routinely changed 2-3 times per week for “static” cages (passive air exchange) or every 2 weeks if combined with individual cage positive ventilation. Current strategies to avoid infections that may affect the health of laboratory mice or compromise scientific data usually include sterilization of mouse cages and accessories (e.g., feed, water, bedding). Plastic or metal cage components are usually sterilized using pressurized steam within large, immobile autoclaves. However, autoclaves involve complicated piping and controls, consume much energy and water, create an uncomfortably humid working environment, and are prone to frequent breakdowns including steam blowouts. The chamber size of such industrial autoclaves is also not compatible with efficient throughput, resulting in batch sizes that are too large to be used quickly and thereby creating unnecessary demands on limited storage space. In addition, repeated autoclaving causes plastic mouse cages to craze (become opaque) and crack, which shortens the useful lifespan of these expensive items. Growing pressures on research and development funding, energy conservation, and occupational safety, while not compromising research results, creates a need for alternative sterilization methods in this sector. While there are other sterilization modalities (e.g., irradiation, chemical sterilants) besides autoclaving, none of them has proven to be practical, affordable, environmentally acceptable and safe.

Thus, there is a need for an improved methods and an improved apparatus for sterilizing animal cages.

### SUMMARY OF THE INVENTION

The present invention provides a method and an apparatus for sterilizing objects, such as animal cages.

In one form, the apparatus of the invention includes a housing that defines a sterilizing chamber for the objects. The apparatus includes a first air inlet in fluid communication with the sterilizing chamber, and a first air outlet in fluid communication with the sterilizing chamber. The first air inlet and the first air outlet are structured to be placed in fluid communication with a source of dry heat. A second air inlet is in fluid communication with the sterilizing chamber and a source of filtered air. A second air outlet is in fluid communication with the sterilizing chamber and an exterior space outside of the sterilizing chamber. Dry heat flows in the first air inlet, over the objects in the sterilizing chamber, and out the first air outlet. Dampers are then closed to seal off the first air inlet and the first air outlet. After sterilization, dampers are opened in the second air inlet and the second air outlet, and cooling filtered air is directed in the second air inlet, over the objects in the sterilizing chamber, and out the second air outlet. Dampers can then be closed to seal off the second air inlet and the second air outlet to avoid contamination of the sterilized objects.

In one version, the method of the invention for sterilizing animal cages uses an apparatus including (i) a housing defining a sterilizing chamber, (ii) an air inlet in fluid communication with the sterilizing chamber, (iii) an air inlet damper having a closed position for preventing flow from the air inlet to the sterilizing chamber, and also having an open position for allowing flow from the air inlet to the sterilizing chamber, (iv) an air outlet in fluid communication with the sterilizing chamber, and (v) an air outlet damper having a closed position for preventing flow from the sterilizing chamber to the air outlet, and also having an open position for allowing flow from the sterilizing chamber to the air outlet. Animal cages to be sterilized are placed in the sterilizing chamber of the apparatus. The air inlet is placed in fluid communication with a source of dry heat, and the air inlet damper is placed in the open position of the air inlet damper. The air outlet is placed in fluid communication with the source of dry heat, and the air outlet damper is placed in the open position of the air outlet damper. Dry heat is then directed through the air inlet, over the cages in the sterilizing chamber, and out the air outlet thereby sterilizing the animal cages. In the method, the apparatus can further include (vi) a source of filtered air, (vii) a second air inlet in fluid communication with the sterilizing chamber and the source of filtered air, and (viii) a second air outlet in fluid communication with the sterilizing chamber and an exterior space outside of the sterilizing chamber. The air inlet damper is placed in the closed position of the air inlet damper, and the air outlet damper is placed in the closed position of the air outlet damper. Filtered air is then directed through the second air inlet, over the cages in the sterilizing chamber, and out the second air outlet thereby cooling the sterilized animal cages.

Dry heat is advantageous over autoclaving because it requires no dedicated plumbing, consumes less energy and no water, has few working parts so maintenance, downtime and repair costs are greatly reduced, extends the useful life of cages and provides a more comfortable environment for staff. By “dry heat”, we mean hot air that is either free from water vapor, or has very little water vapor, and where moisture plays a minimal or no role in the process of sterilization.

Several additional features of this invention make it even more attractive for sterilizing animal cages: (1) a smaller size and modularity permit easier installation and use (e.g., fits through normal doorways, uses conventional power outlets, simple to replace or expand capacity with multiple units so one only has to purchase the capacity needed, smaller batch sizes for more efficient throughput); (2) its mobility allows for sterilization at the location of use, particularly useful in decontaminating biohazardous or infectious materials that otherwise have to be transported to a centrally installed autoclave; (3) inner containers that hold cage components avoid the need for further wrapping or packaging, and can be stored or transported to adjacent rooms while protecting the contents from contamination, providing greater flexibility of use; (4) forced high efficiency particulate air (HEPA)-filtered air cools down sterilized items faster for greater safety and productivity, and keeps the contents sterile when the container is opened to remove them.

In the sterilization apparatus of the present invention, the heat source section (which can be mobile or non-mobile) is smaller than any autoclaves designed for large volume rodent cage sterilization, thereby avoiding major renovation costs to install or replace large stainless steel autoclave chambers that require extensive fittings and controls to contain pressurized steam. The mobile section of the sterilization apparatus that holds the cages allows for more flexibility and productivity in processing sterilized items so that output can more closely match demand on an hourly basis rather than inefficiently filling up a large space with a maximum load. Also, the mobile sections of the sterilization apparatus can be wheeled to the room(s) where the animal cages are needed while cooling along the way, to save more time. In addition, each mobile unit takes up a smaller footprint which is important in congested hallways. The mobile sections of the sterilizer are sufficiently insulated so their outside surface remains cool to the touch whether or not they are attached to the heating unit, to provide a safer and more comfortable work place. An alternative embodiment combines the sterilization chamber, blower, filters, and heat source within the same mobile apparatus. When the source of dry heat is contained within the same apparatus as the blower, filter, and sterilization chamber, even more flexibility and economy in routine use is provided. This version is especially appropriate for use in smaller animal facilities or for animal care operations in which further modularity is indicated.

The modular dry heat sterilizing apparatus of the invention provides a mobile and modular unit that provides more flexibility in terms of cheaper unit costs, modularity/multiplicity that can accommodate animal facilities of more varied sizes and more varied needs (e.g., a BL-2 suite that may require more frequent sterilization runs for decontamination of biohazardous cages and other materials and that may be far removed from the central cage processing area).

In the present invention, the HEPA filtration component ensures sterility of a microisolator cage unit or related supplies until those articles are delivered to and used at the actual animal housing site. This is advantageous as both gnotobiotic as well as conventional specific-pathogen-free rodent facilities sterilize caging and other components (e.g., feed, water bottles) prior to their use. Thus, HEPA-filtered air within the chamber of the invention is one feature. A flexible manufacturing approach can provide versions with and without to the both the lab animal and private veterinary practice markets.

Current sterilizers used in lab animal care are all large, permanently installed steam autoclaves, contrasting in several aspects to the present invention. These steam autoclaves are priced at tens to hundreds of thousands of dollars, excluding often substantial installation costs such as additional steam supply and exhaust if the unit is retrofitted within an existing vivarium. By comparison, the present invention has a price point significantly less, with plug-and-play flexibility, lower operating costs, and a safer and more comfortable work environment for employees.

These and other features, aspects, and advantages of the present invention will become better understood upon consideration of the following detailed description, drawings and appended claims.

### BRIEF DESCRIPTION OF THE DRAWINGS

FIG. 1 is a front elevational view of a modular dry heat sterilizing apparatus according to the invention.

FIG. 2 is a rear elevational view of the modular dry heat sterilizing apparatus of FIG. 1.

FIG. 3 is a left side elevational view of the modular dry heat sterilizing apparatus of FIG. 1.

FIG. 4 is a top plan view of the modular dry heat sterilizing apparatus of FIG. 1.

FIG. 5 is a cross-sectional view taken along line 5-5 of FIG. 4.

FIG. 6 is a detailed view taken at line 6-6 of FIG. 5 showing an alternative version of a modular dry heat sterilizing apparatus according to the invention.

Like reference numerals will be used to refer to like parts from Figure to Figure in the following description of the drawings.

### DETAILED DESCRIPTION OF THE INVENTION

A device is described herein that uses convection dry heat to sterilize laboratory animal (e.g., rodent) cages or any other solid objects. Example animal cages can be found in U.S. Pat. Nos. 4,640,228 and 4,480,587 to Sedlacek et al. which are incorporated herein by reference. The size of the device is designed to provide approximately an hour's worth of work (e.g., 64 bedded cage bottoms). A protective container that holds the cages is another important optional component of this invention, to maintain sterility of clean cages after exiting the sterilizer until those cages are used. The device also provides for containment of effluent from dirty cages that must be sterilized to destroy biohazards or rodent pathogens prior to washing and reuse. The device improves vivarium workforce productivity and rodent pathogen control.

In one embodiment, the invention provides a modular unit for sterilizing animal (e.g., mouse) cage components via convective dry heat that is mobile, fits through normal doorways and only requires an electrical connection for use. Each unit can be sized to hold a batch of materials to supply an animal care technician 45-90 minutes worth of work (e.g., approximately 64-72 bedded shoebox-sized mouse cage bottoms). Cages are in a tightly closed sterilizing chamber with an air inlet and an air outlet on its top and bottom. These inlets are closed except when they connect with a blower driving a dry heat source, circulating preferably from bottom to top of the sterilizing chamber. The unit can have a heating range of 260-400° F. that circulates dry heat over the cages until they are sterilized. After sterilization is completed, the air inlet and outlet connections of the sterilizing chamber are closed and the sterilizing chamber, containing the sterile cages, is removed from the dry heat source. This sterilizing chamber may be aseptically connected to pressurized HEPA-filtered air to accelerate cooling, after which it can be transported to a point of use or stored for later use without compromising the sterility of the cages. When the cages are ready for use, the HEPA-filtered air can be turned on to maintain a positive pressure before the door to the sterilizing chamber is opened in order to prevent introduction of potentially contaminating air.

In another example embodiment, the invention provides a modular unit for sterilizing animal components incorporating a small mobile insulated docking cage pod (56-72 cages, 400 pounds or less, mobile (for example on casters), suitable to fit through door openings (for example less than 40 inches wide and less than 80 inches tall), having a fan on the mobile pod for continuous ventilation and maintaining of static pressure, manual dampers for docking and disengagement when the sterilizing cycle is complete and a stationary conditioning air system that remains continuously on at an operating temperature of 260-400° F. for docking with the cage pods. Some advantageous attributes of this totally self contained unit are the mobility of the small insulated pods in combination with the stationary conditioning air system remaining continuously at temperature before, during and after the cage pod is removed.

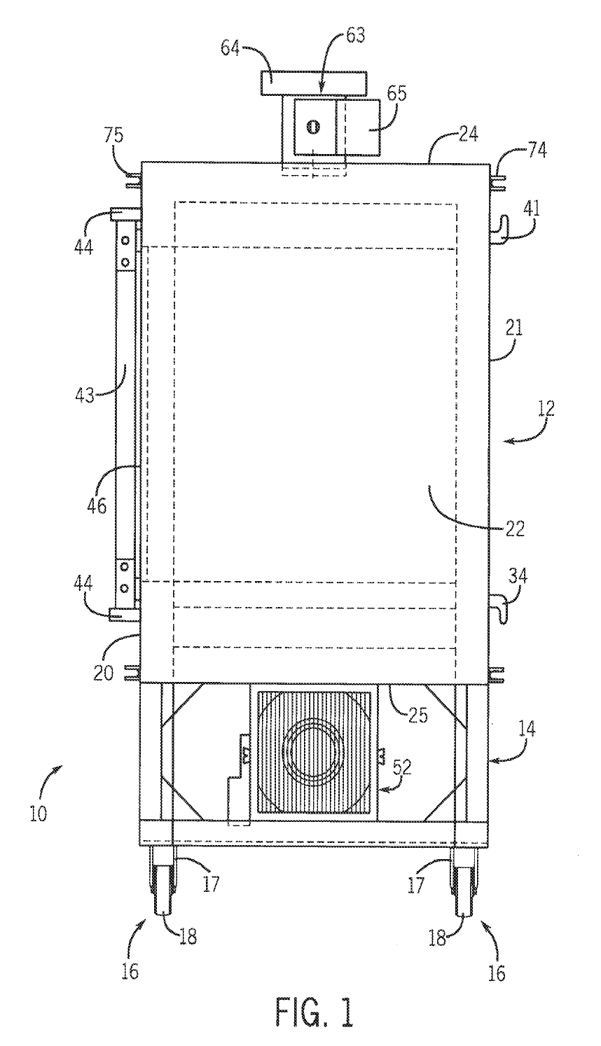

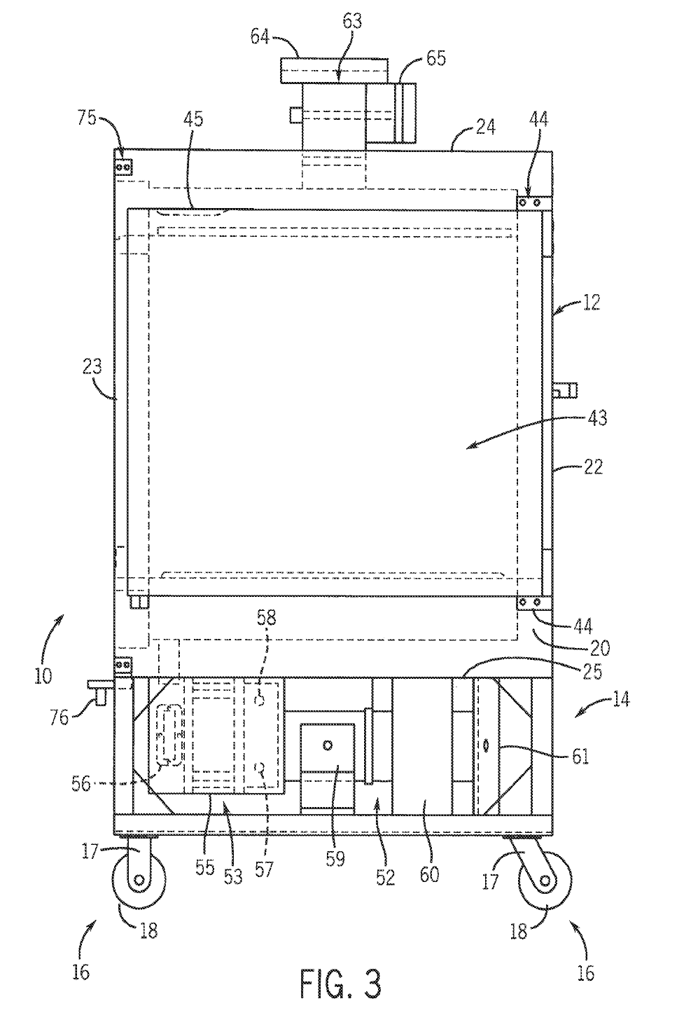

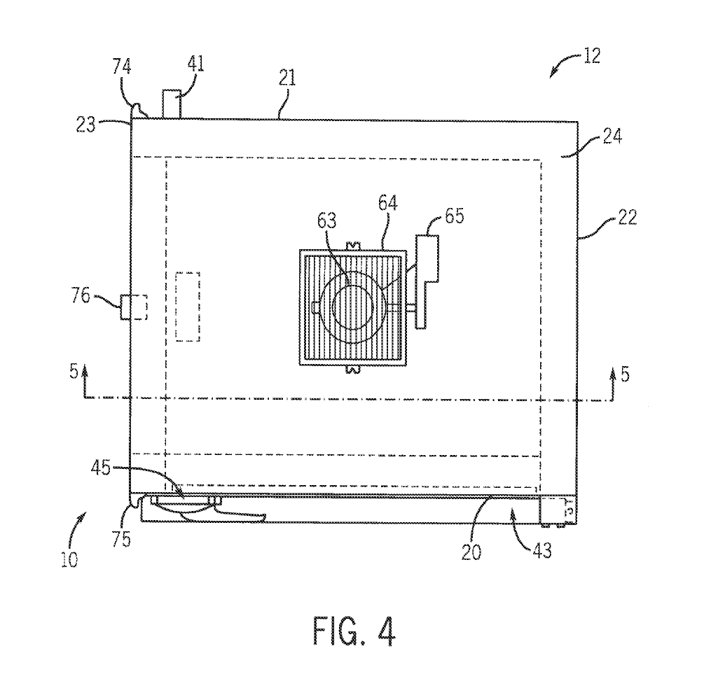

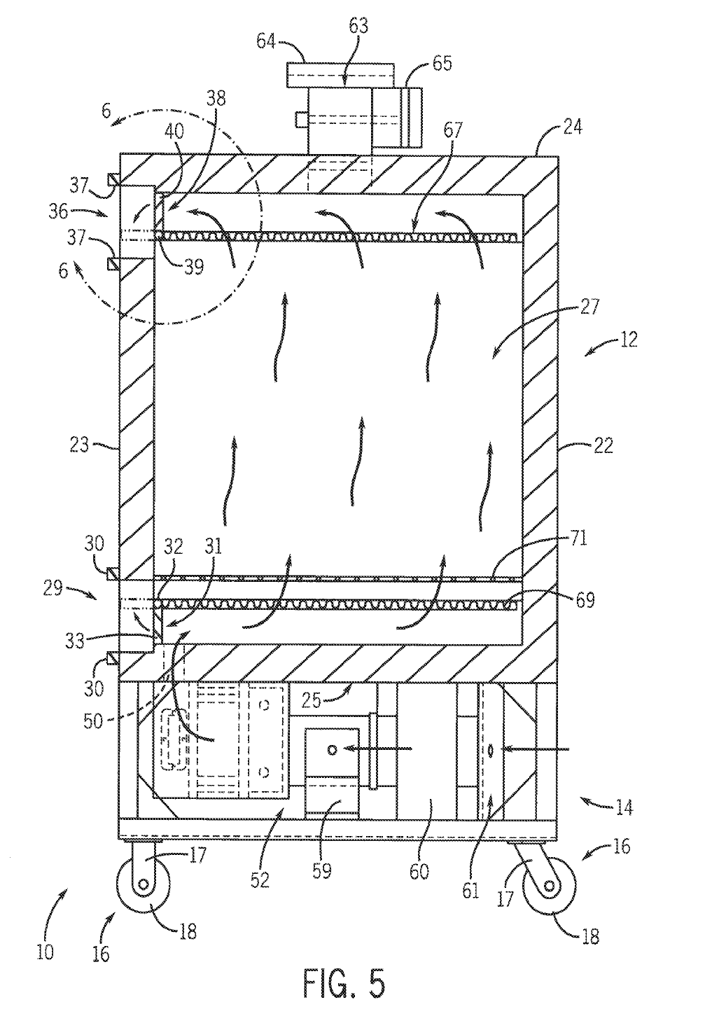

Turning now to FIGS. 1-5, there is shown a non-limiting example embodiment of a modular dry heat sterilizing apparatus 10 according to the invention. The apparatus 10 includes a housing 12 mounted on a frame 14. At the bottom of the frame 14, there are casters 16 comprised of a fork 17 and a rotating wheel 18. The housing 12 has a left side wall 20, a right side wall 21, a front wall 22, a rear wall 23, a top wall 24 and a bottom wall 25 that define an interior sterilizing chamber 27 of the apparatus 10 (see FIG. 5). The housing 12 can be formed with an aluminum exterior and interior around a middle insulation blanket. Other materials are also suitable for the elevated temperatures of the housing 12.

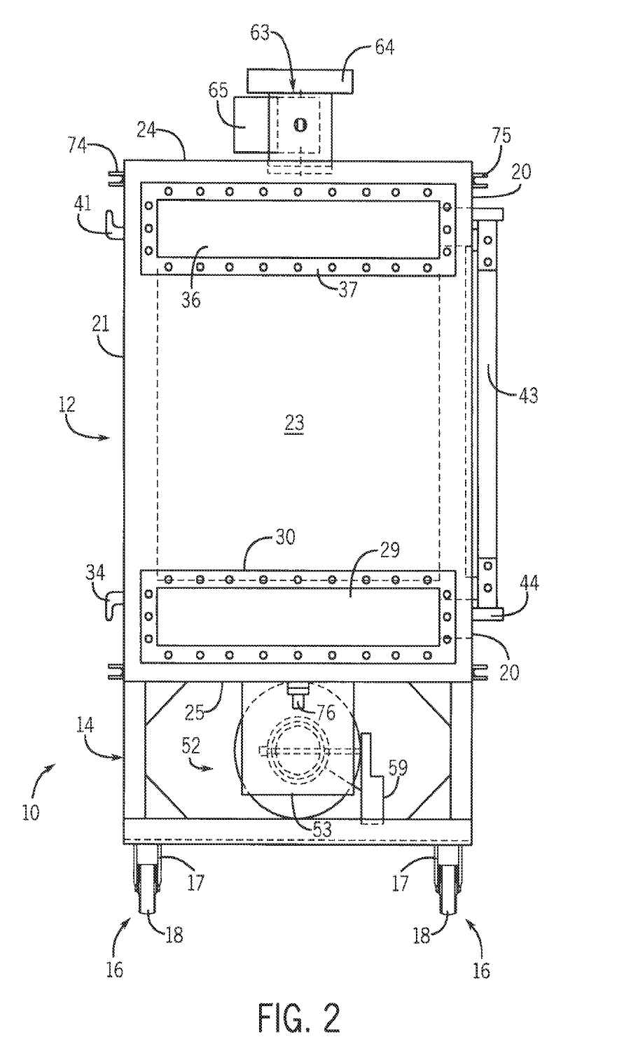

The rear wall 23 of the apparatus 10 has a rectangular dry heat inlet 29 surrounded by a sealing gasket 30. Silicone materials are suitable for the sealing gasket 30 and other similar gaskets in the apparatus 10. Looking at FIG. 5, a damper 31 having a rotatable shaft 32 and an attached plate 33 is arranged adjacent the dry heat inlet 29. The shaft 32 terminates in a handle 34 that protrudes outward from the right side wall 21 (see FIG. 2). The damper 31 has a closed position for preventing flow from the dry heat inlet 29 to the sterilizing chamber 27. This closed position is shown in full lines in FIG. 5. The damper 31 also has an open position for allowing flow from the dry heat inlet 29 to the sterilizing chamber 27. This open position is shown in phantom lines in FIG. 5. The damper 31 can be moved from the closed position to the open position by rotating the handle 34 such that the plate 33 moves in direction U in FIG. 5. The damper 31 can be moved from the open position to the closed position by rotating the handle 34 such that the plate 33 moves in the direction opposite direction U in FIG. 5. While manual movement of the damper 31 by handle 34 is shown in this version of the apparatus 10, the damper 31 can also be moved by a suitable damper motor under electronic control.

The rear wall 23 of the apparatus 10 also has a rectangular dry heat outlet 36 surrounded by a sealing gasket 37. Looking at FIG. 5, a damper 38 having a rotatable shaft 39 and an attached plate 40 is arranged adjacent the dry heat outlet 36. The shaft 39 terminates in a handle 41 that protrudes outward from the right side wall 21 (see FIG. 2). The damper 38 has a closed position for preventing flow from the dry heat outlet 36 to the sterilizing chamber 27. This closed position is shown in full lines in FIG. 5. The damper 38 also has an open position for allowing flow from the dry heat outlet 36 to the sterilizing chamber 27. This open position is shown in phantom lines in FIG. 5. The damper 38 can be moved from the closed position to the open position by rotating the handle 41 such that the plate 40 moves in direction D in FIG. 5. The damper 38 can be moved from the open position to the closed position by rotating the handle 41 such that the plate 40 moves in the direction opposite direction D in FIG. 5. While manual movement of the damper 38 by handle 41 is shown in this version of the apparatus 10, the damper 38 can also be moved by a suitable damper motor under electronic control. This could also be accomplished via a spring loaded damper.

The left side wall 20 of the apparatus 10 has a door 43 providing access to the sterilizing chamber 27. The door 43 rotates on hinges 44 and is secured to the left side wall 20 by way of a latch 45. A gasket 46 provides a tight seal between the door 43 and the left side wall 20. Optionally, the apparatus 10 includes a second door reducing the space needed within an animal room and reducing the volume of turbulence when the door is opened.

The bottom wall 25 of the apparatus 10 has a filtered air inlet 50 that is in fluid communication with a source of filtered air 52. The source of filtered air 52 includes a filter box 53 housing a HEPA filter. An access panel 55 allows the user to change the HEPA filter. One non-limiting example HEPA filter is an 8″×8″×3¾″ HEPA filter, 99.97% eff. @ 0.3 microns. Ports 56, 57 and 58 allow for an in-place method for evaluating the HEPA filtration. The source of filtered air 52 includes a damper 59. The damper 59 has a closed position for preventing flow from the source of filtered air 52 to the sterilizing chamber 27, and the damper 59 also has an open position for allowing flow from the source of filtered air 52 to the sterilizing chamber 27. The source of filtered air 52 includes a fan 60 and an air intake filter 61. A non-limiting example fan 60 has an inline duct, 6″ diameter, plastic housing, with 115 volt, 60 hz, totally enclosed air over motor.

The top wall 24 of the apparatus 10 includes a filtered air outlet 63 having a filter 64 and a damper 65. A non-limiting example filter 64 is a 10″×10″×1″ aluminum mesh. An electronic controller of the apparatus 10 includes a setting that turns on the fan 60, moves the damper 59 to the open position of the damper 59, and moves the damper 65 to the open position of the damper 65. The dampers 59 and 65 can be moved by a suitable damper motor under electronic control of the controller of the apparatus 10. The controller of the apparatus 10 receives AC electrical power (such as from an AC power outlet) or DC electrical power (such as from a battery) and transmits AC or DC electrical power to the fan 60 and dampers 59 and 65 in a conventional manner.

Looking at FIG. 5, the sterilizing chamber 27 of the apparatus 10 has a top diffuser panel 67 and a bottom diffuser panel 69 that act as flow regulators in the sterilizing chamber 27. The top diffuser panel 67 and the bottom diffuser panel 69 laterally span the sterilizing chamber 27 of the apparatus 10, and can comprise aluminum with semi-pierce adjustable louvers. A wire rack 71 can be included in the sterilizing chamber 27 of the apparatus 10. The wire rack 71 provides a flow thorough surface for arranging the animal cages to be sterilized. Optionally, a removable container having an outer wall with throughholes can contain the animal cages to be sterilized on the wire rack 71 in the sterilizing chamber 27. In one non-limiting embodiment, the sterilizing chamber 27 measures 26 inches wide, 35 inches deep and 30 inches between the top diffuser panel 67 and the bottom diffuser panel 69. This non-limiting example apparatus can have 6 inches between the top diffuser panel 67 and the top wall 24, six inches between the bottom diffuser panel 69 and the bottom wall 25, forty-six inches overall depth, seventy-five inches overall height, and twenty-seven inches overall width.

The apparatus 10 can receive dry heat from a non-mobile or mobile source of dry heat as follows. A source of dry heat and a blower can be provided in a dry heat unit separate from the apparatus 10. A dry heat supply duct of the dry heat unit is placed in fluid communication with the dry heat inlet 29 of the apparatus 10. The gasket 30 provides a fluid tight fit between the dry heat supply duct of the dry heat unit and the dry heat inlet 29, and locating pins can be provided in or adjacent the gasket 30. Likewise, a dry heat return duct of the dry heat unit is placed in fluid communication with the dry heat outlet 36 of the apparatus 10. The gasket 37 provides a fluid tight fit between the dry heat return duct of the dry heat unit and the dry heat outlet 36 and locating pins can be provided in or adjacent the gasket 37. Suitable latching components can be arranged on the apparatus 10 and the dry heat unit to keep the apparatus 10 and the dry heat unit latched together. For example, catch 74 and catch 75 on the apparatus 10 can engage associated latches on the dry heat unit. Alignment of the apparatus 10 and the dry heat unit can be facilitated by a guide pin 76 that mates with a locating element (such as a V-shaped block) on the dry heat unit.

After the dry heat supply duct of the dry heat unit is placed in fluid communication with the dry heat inlet 29 and the dry heat return duct of the dry heat unit is placed in fluid communication with the dry heat outlet 36, the user can rotate the handle 34 such that the plate 33 of the damper 31 moves in the open position shown in phantom lines in FIG. 5. Likewise, the user can rotate the handle 41 such that the plate 40 of the damper 38 moves in the open position shown in phantom lines in FIG. 5. The heating unit and the blower of the dry heat unit are then activated such that dry heat is directed through the dry heat inlet 29, over the cages in the sterilizing chamber 27, and out the dry heat outlet 36. FIG. 5 includes arrows showing the flow directions of the dry heat. A non-limiting example for the dry heat flow is up to 1200 scfm @ 1.0″ sp @ 70° F. A suitable timer on the dry heat unit can be used to insure sufficient contact time for the cages and dry heat such that sufficient sterilization has occurred.

After the cages have been sterilized, the user can rotate the handle 34 such that the plate 33 of the damper 31 moves in the closed position shown in full lines in FIG. 5. Also, the user can rotate the handle 41 such that the plate 40 of the damper 38 moves in the closed position shown in full lines in FIG. 5. The user can then choose a controller ‘on’ setting on the apparatus 10 that turns on the fan 60, moves the damper 59 to the open position of the damper 59, and moves the damper 65 to the open position of the damper 65. Filtered cooling air enters the filtered air inlet 50 and moves over the cages to cool the cages in the sterilizing chamber 27. The filtered air exits the sterilizing chamber 27 through the filtered air outlet 63. The user can then choose the controller ‘off’ setting that turns off the fan 60, moves the damper 59 to the closed position of the damper 59, and moves the damper 65 to the closed position of the damper 65. A non-limiting example for the filtered air flow is approximately 167 scfm @ 0.6″ sp @ 70° F. After cooling the cages in the sterilizing chamber 27, the apparatus 10 can be transported to a point of use or stored for later use without compromising the sterility of the cages. When the cages are ready for use, the HEPA-filtered air can be turned on to maintain a positive pressure before the door 43 to the sterilizing chamber 27 is opened in order to prevent introduction of potentially contaminating air.

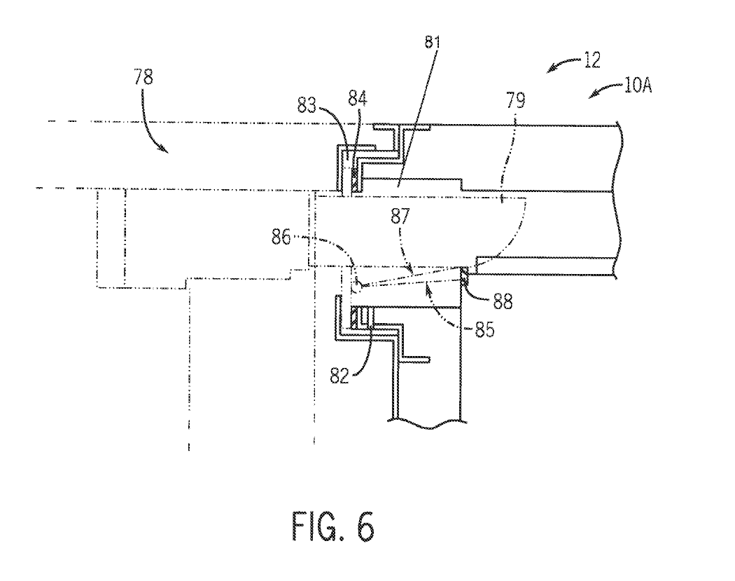

Turning now to FIG. 6, there is shown an alternative structure for placing the dry heat return duct of the dry heat unit in fluid communication with the dry heat outlet. In FIG. 6, the source of dry heat 78 includes a protruding dry heat return duct 79. A dry heat outlet 81 with a protruding port 82 is provided on the apparatus 10A. The protruding port 82 on the apparatus 10A is received in a recess 83 of the source of dry heat 78, and a sealing gasket 84 insures a fluid tight fit. When the protruding port 82 on the apparatus 10A is received in a recess 83 of the source of dry heat 78, a section of the dry heat return duct 79 opens a damper 85 by rotating plate 87 on hinge 86 to the position shown in FIG. 6. A sealing gasket 88 provides a fluid tight fit at the bottom of the plate 87. Suitable latching components can be arranged on the apparatus 10A and the source of dry heat 78 to keep the apparatus 10A and the source of dry heat 78 latched together. The same type of structure as shown in FIG. 6 can be used for placing the dry heat supply duct of the dry heat unit in fluid communication with the dry heat inlet of the apparatus 10A.

Other variations for the apparatus 10 and the apparatus 10A are possible. For example, the dry heat could flow from top to bottom in the sterilizing chamber 27. Also, the dry heat unit including a source of dry heat and a blower can be integrated into the housing 12 or otherwise mounted on the frame 14.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

### INDUSTRIAL APPLICABILITY

The invention relates to a method and an apparatus for sterilizing objects, and in particular, the invention relates to a modular dry heat sterilizer for animal cages.

1. An apparatus for sterilizing objects, the apparatus comprising:
a housing defining a sterilizing chamber;
a first air inlet in fluid communication with the sterilizing chamber, the first air inlet being structured to be placed in fluid communication with a source of dry heat;
a first air inlet damper having a closed position for preventing flow from the first air inlet to the sterilizing chamber, the first air inlet damper also having an open position for allowing flow from the first air inlet to the sterilizing chamber;
a first air outlet in fluid communication with the sterilizing chamber, the first air outlet being structured to be placed in fluid communication with the source of dry heat;
a first air outlet damper having a closed position for preventing flow from the sterilizing chamber to the first air outlet, the first air outlet damper also having an open position for allowing flow from the sterilizing chamber to the first air outlet;
a source of filtered air;
a second air inlet in fluid communication with the sterilizing chamber and the source of filtered air;
a second air inlet damper having a closed position for preventing flow from the source of filtered air to the sterilizing chamber, the second air inlet damper also having an open position for allowing flow from the source of filtered air to the sterilizing chamber;
a second air outlet in fluid communication with the sterilizing chamber and an exterior space outside of the sterilizing chamber; and
a second air outlet damper having a closed position for preventing flow from the sterilizing chamber to the exterior space, the second air outlet damper also having an open position for allowing flow from the sterilizing chamber to the exterior space.

2. The apparatus of claim 1 wherein:
the apparatus includes the source of dry heat.

3. The apparatus of claim 2 wherein:
the apparatus includes a blower for circulating the dry heat through the first air inlet, through the sterilizing chamber, and through the first air outlet.

4. The apparatus of claim 3 wherein:
the source of dry heat and the blower are provided in a dry heat unit separate from the housing.

5. The apparatus of claim 3 wherein:
the source of dry heat is contained within the same apparatus as the blower, filter, and sterilization chamber
the first air inlet, the first air outlet, the second air inlet, and the second air outlet are located in the housing, and
the housing, the source of filtered air, the source of dry heat, and the blower are mounted on a movable frame.

6. The apparatus of claim 1 wherein:
the source of filtered air comprises a high efficiency particulate air filter and a fan for forcing air through the air filter, through the second air inlet, through the sterilizing chamber, and through the second air outlet.

7. The apparatus of claim 6 wherein:
the apparatus includes an electrical controller,
the second air inlet damper includes a first actuator for moving the second air inlet damper between the closed position of the second air inlet damper and the open position of the second air inlet damper, the first actuator being in electrical communication with the controller,
the second air outlet damper includes a second actuator for moving the second air outlet damper between the closed position of the second air outlet damper and the open position of the second air outlet damper, the second actuator being in electrical communication with the controller,
the fan is in electrical communication with the controller, and
the controller includes a setting that turns on the fan, moves the second air inlet damper to the open position of the second air inlet damper, and moves the second air outlet damper to the open position of the second air outlet damper.

8. The apparatus of claim 1 wherein:
the source of dry heat and the blower are provided in a dry heat unit separate from the housing,
the first air inlet is dimensioned to sealingly engage a dry heat supply duct of the dry heat unit, and
the first air outlet is dimensioned to sealingly engage a dry heat return duct of the dry heat unit.

9. (canceled)

10. (canceled)

11. (canceled)

12. The apparatus of claim 1 wherein:
the first air inlet and the first air outlet are located in a wall of the housing.

13. (canceled)

14. The apparatus of claim 1 wherein:
the second air inlet and the second air outlet are located in opposite walls of the housing.

15. The apparatus of claim 1 wherein:
the first air inlet, the first air outlet, the second air inlet, and the second air outlet are located in the housing, and
the housing and the source of filtered air are assembled to a movable frame.

16. (canceled)

17. The apparatus of claim 1 wherein:
the first air inlet and the first air outlet are located in a first side wall of the housing,
the second air inlet is located in a bottom wall of the housing,
the second air outlet is located in a top wall of the housing, and
the housing and the source of filtered air are assembled to a movable frame.

18. (canceled)

19. (canceled)

20. The apparatus of claim 1 wherein:
the first air inlet damper includes a first handle for moving the first air inlet damper between the closed position of the first air inlet damper and the open position of the first air inlet damper, and
the first air outlet damper includes a second handle for moving the first air outlet damper between the closed position of the first air outlet damper and the open position of the first air outlet damper.

21. The apparatus of claim 1 further comprising:
a flow regulator positioned in a flow path between a central section of the sterilizing chamber and the first air outlet damper.

22. The apparatus of claim 1 further comprising:
a flow regulator positioned in a flow path between the first air inlet damper and a central section of the sterilizing chamber.

23. (canceled)

24. A method for sterilizing animal cages, the method comprising:
(a) providing an apparatus including (i) a housing defining a sterilizing chamber, (ii) an air inlet in fluid communication with the sterilizing chamber, (iii) an air inlet damper having a closed position for preventing flow from the air inlet to the sterilizing chamber, and also having an open position for allowing flow from the air inlet to the sterilizing chamber, (iv) an air outlet in fluid communication with the sterilizing chamber, and (v) an air outlet damper having a closed position for preventing flow from the sterilizing chamber to the air outlet, and also having an open position for allowing flow from the sterilizing chamber to the air outlet;
(b) placing animal cages to be sterilized in the sterilizing chamber;
(c) placing the air inlet in fluid communication with a source of dry heat;
(d) placing the air inlet damper in the open position of the air inlet damper;
(e) placing the air outlet in fluid communication with the source of dry heat;
(f) placing the air outlet damper in the open position of the air outlet damper; and
(g) directing the dry heat through the air inlet, over the cages in the sterilizing chamber, and out the air outlet.

25. The method of claim 24 wherein:
the apparatus further includes (vi) a source of filtered air, (vii) a second air inlet in fluid communication with the sterilizing chamber and the source of filtered air, and (viii) a second air outlet in fluid communication with the sterilizing chamber and an exterior space outside of the sterilizing chamber, and
the method further comprises
(h) placing the air inlet damper in the closed position of the air inlet damper;
(i) placing the air outlet damper in the closed position of the air outlet damper, and
(j) directing the filtered air through the second air inlet, over the cages in the sterilizing chamber, and out the second air outlet.

26. The method of claim 25 wherein:
the apparatus includes (ix) a second air inlet damper having a closed position for preventing flow from the source of filtered air to the sterilizing chamber, and also having an open position for allowing flow from the source of filtered air to the sterilizing chamber, and (x) a second air outlet damper having a closed position for preventing flow from the sterilizing chamber to the exterior space, and also having an open position for allowing flow from the sterilizing chamber to the exterior space, and
step (j) further comprises placing the second air inlet damper in the open position of the second air inlet damper and placing the second air outlet damper in the open position of the second air outlet damper before directing the filtered air through the second air inlet, over the cages in the sterilizing chamber, and out the second air outlet.

27. The method of claim 26 wherein:
step (j) further comprises placing the second air inlet damper in the closed position of the second air inlet damper and placing the second air outlet damper in the closed position of the second air outlet damper after directing the filtered air through the second air inlet, over the cages in the sterilizing chamber, and out the second air outlet.

28. (canceled)

29. (canceled)

30. The method of claim 24 wherein step (b) comprises:
placing the animal cages in a removable container having an outer wall with throughholes, the container being dimensioned to fit within the sterilizing chamber, and
placing the removable container in the sterilizing chamber.
